(19) United States
(12) Patent Application Publication
(10) Pub. No.: US 2025/0064342 A1
(43) Pub. Date: Feb. 27, 2025

(54) INJURY MONITORING SYSTEM

(71) Applicant: Athletic Impact Analysis, LLC, Rawlins, WY (US)

(72) Inventors: Bruce Furst, Austin, TX (US); John Cronin, Jericho, VT (US); Michael D’Andrea, Burlington, VT (US); Michael Baker, Georgia, VT (US)

(21) Appl. No.: 18/117,730

(22) Filed: Mar. 6, 2023

Related U.S. Application Data
(60) Provisional application No. 63/316,884, filed on Mar. 4, 2022.

(51) Int. Cl. A61B 5/11; A61B 5/00; G16H 10/60; G16H 50/30

(52) U.S. Cl. CPC A61B 5/1114; A61B 5/0077; G16H 50/30; A61B 5/6803; A61B 5/746; G16H 10/60

(57) ABSTRACT

A system and method for monitoring the health and injury risk of players of a sport, particularly where physical contact occurs, using image and sensor data from sensors worn by the players. An injury risk score may be determined for each of the camera data and the sensor data. The data is then correlated to determine a risk score that may comprise a likelihood of the player having sustained an injury or the player's susceptibility to sustaining a future injury.

### CROSS-REFERENCE TO RELATED APPLICATION

This application claims the priority benefit of U.S. provisional patent application 63/316,884 filed Mar. 4, 2022, which is incorporated by reference herein in its entirety.

### FIELD OF THE DISCLOSURE

The present disclosure is generally related to collecting player data and, more specifically, relates to a system for evaluating a player's injury risk based on the collected player data.

### BACKGROUND

Wearable sensors are used to acquire a wide range of information about the user wearing the sensors. Examples of wearable sensors include accelerometers, gyroscopes, compasses, global positioning systems (GPS), heart rate monitors, and electromyography sensors. In addition, wearable location and health-based sensors can acquire a range of different information about the wearer, which can monitor the user's condition.

Wearable sensors can be affixed to a player participating in a sport to acquire information about the player. This is particularly useful in sports where physical contact may be experienced by a player, such as in American football. Wearable sensors can be used to determine the intensity, direction, and location of an impact received by the player. The information can be compiled into graphs for viewing or analysis to determine the player's risk of developing an injury both in the short and long term.

Sports with physical contact, such as American football, can be taxing on players' bodies. Some monitoring methods include affixing accelerometers to the player, such as in a helmet, and using data collected during practice or gameplay to determine the severity and frequency of impacts. When correlated with reported injuries, this impact data may be used to predict future chronic injuries, which may inform decisions to manage player health or changes to protective equipment or game rules to improve player health and safety.

Additional methods of monitoring a patient comprise using video data to infer the forces imparted during impacts experienced by the player. While less costly to acquire and more readily available, this data may be less reliable as it may not be unable to accurately account for players' body mass or correctly identify the exact location of impact. Therefore, a system is desired which can improve the accuracy of a video-based player monitoring system.

A method of alerting a player's coach of an impact location or severity that may indicate an injury that even the player may be unaware of is desired. Players may sustain an injury but not immediately feel the pain or become weakened and more prone to injury without awareness of their increased risk. A method of assessing such increase in risk, notifying staff, and allowing for a decision to be made to prevent injury to the player is desired.

A system and method for monitoring the health and injury risk of players of a sport, particularly where physical contact occurs, using image and sensor data from sensors worn by the players. An injury risk score may be determined for each of the camera data and the sensor data. The data is then correlated to determine a risk score that may comprise a likelihood of the player having sustained an injury or the player's susceptibility to sustaining a future injury.

### DESCRIPTIONS OF THE DRAWINGS

FIG. 1: illustrates an injury monitoring system, according to an embodiment.

FIG. 2: illustrates a sensor database, according to an embodiment.

FIG. 3: illustrates a camera database, according to an embodiment.

FIG. 4: illustrates a correlation database, according to an embodiment.

FIG. 5: illustrates a base module, according to an embodiment.

FIG. 6: illustrates a sensor prediction module, according to an embodiment.

FIG. 7: illustrates a camera prediction module, according to an embodiment.

FIG. 8: illustrates a correlation module, according to an embodiment.

### DETAILED DESCRIPTION

Embodiments of the present disclosure will be described more fully hereinafter with reference to the accompanying drawings in which like numerals represent like elements throughout the several figures and in which example embodiments are shown. Embodiments of the claims may, however, be embodied in many different forms and should not be construed as limited to the embodiments set forth herein. The examples set forth herein are non-limiting examples and are merely examples among other possible examples.

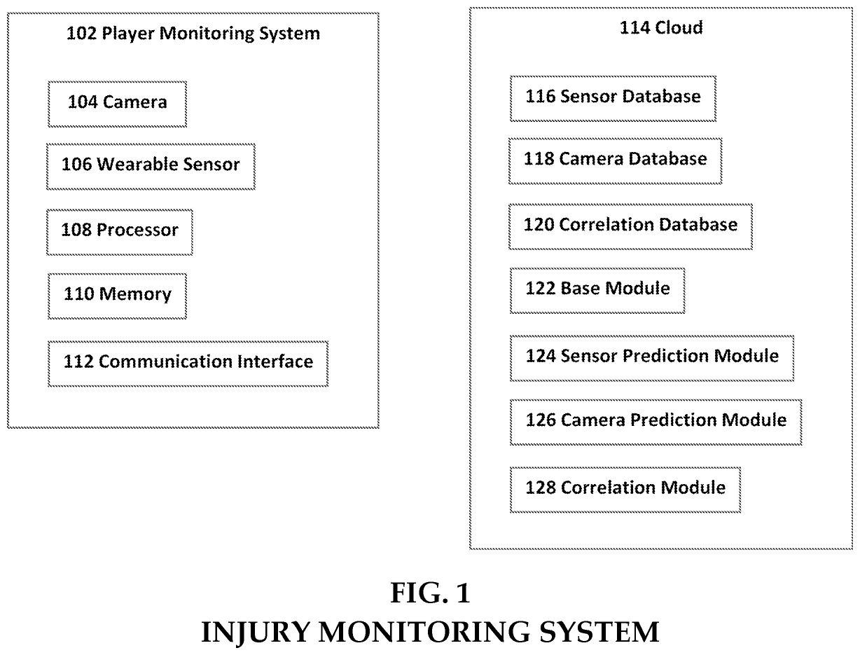

FIG. 1 is an injury monitoring system. This system comprises a player monitoring system 102, which is a system comprised of at least one camera 104, at least one sensor 106, a processor 108, a memory 110, and a communications interface 112. The player monitoring system 102 may communicate with a cloud 114 via the communication interface 112. The player monitoring system may operate a base module 114, which may further comprise several base modules, to collect data from at least one camera 104 and the at least one sensor 106 and predict based upon the collected data whether at least one player has sustained an injury or is at increased risk of injury resulting from physical activity such as a sport where physical contact occurs.

A camera 104 is an imaging device for recording visual images. A camera may record single images or a series of images that may comprise a video. Each image comprises an array of pixels that may be represented by a single value, resulting in a monochrome or grayscale image, or may be represented by a color model representing colors. The color model may be any of an RGB or red-green-blue color model, HSL or hue-saturation-lightness model, HSV or hue-saturation-value model, HSI or hue-saturation-intensity model, etc.

A wearable sensor 106 is a sensor that may be affixed to a person either directly, such as by using an adhesive, strap, or band, or may be integrated into an article worn by the person, such as a shirt, socks, undergarments, pants, hat, helmet, etc. In addition, a wearable sensor 106 may include any of an accelerometer, force transducer, gyroscope, compass, a global positioning system (GPS), heart rate monitor, electromyography sensor, etc. A wearable sensor 106 may further be any device worn by a person and collects or transmits data about the person wearing the sensor or the environment surrounding the person.

A processor 108 or controller is a computing device that executes the instructions that comprise a program. The processor 108 may perform operations including basic arithmetic, logic, controlling, and input/output operations based on the instructions it is provided. The processor 108 may further receive data from a memory 110 or database and perform calculations using the received data. The data results may be saved to the memory 110 or a database. The processor 108 may further use a communication interface 112 to communicate with external devices, including a camera 104, wearable device 106, or a cloud 114.

A memory 110 is a data storage device. The data storage device may be comprised of temporary, volatile storage, long-term persistent storage, or a combination of both volatile and non-volatile storage. An example of temporary storage is random access memory (RAM) which is used by the processor 108 to store temporary data while performing the instructions provided by a program. Examples of persistent, non-volatile memory include hard disk drive (HDD), solid-state drive (SSD), flash drives, memory cards, etc. Memory 110 may be located local to a processor 108 or may be located externally, such as in a cloud 114. Memory 110 may additionally store programs to be accessed and executed by a processor 108.

A communication interface 112 may comprise a physical connection or a wireless terminal that allows data to be sent and received between devices. An example of a physical communication interface 112 is an ethernet port, which may be connected to a network switch, router, modem, or directly to another computing device to allow communication between the local device and the devices to which the local device is connected via the communication interface 112. Wireless communication interfaces 112 include Wi-Fi, Bluetooth, Zigbee, 4G, 5G, LTE, etc., and comprise a transceiver and an antenna for sending and receiving data wireless between devices. A communication interface 112 may allow a device to connect to and communicate with a cloud 114.

A cloud 114 is a distributed network comprised of computing and data storage resources. A cloud 114 may be a public cloud, where resources are shared among a large number of unrelated entities via the internet, or a private cloud, where resources are owned by the organization utilizing them and are not shared with other entities. Data in a private cloud may be isolated from the internet or may be encrypted. A cloud 114 is characterized by its redundancy and scalability, where there is no single point of failure, and computing and data storage resources may be requisitioned when needed and repurposed when no longer necessary.

A sensor database 116 stores data collected from one or more wearable sensors 106. Examples of the data stored may be position and orientation data from an accelerometer, GPS, compass, etc., or force data from transducers or strain gages. In addition, the data may include the player wearing the sensor, the sensor type, sensor location, and measurement from the sensor at a specific time. The sensor database 116 may additionally include the type of event and players.

A camera database 118 stores data collected from one or more cameras 104 oriented toward the field of play. At least one camera 104 capturing at least one player. The camera database 118 is comprised of images or video segments, each having a timestamp of when the image or video was acquired. Each image or video segment may be stored as a file.

The correlation database 120 stores correlation data calculated by the correlation module 128. The correlation data comprises the relationship between predictions made by the sensor prediction module 124 using data from at least one wearable sensor 106 and the camera prediction module 126 using data from at least one camera 104.

The base module 122 initializes one or more wearable sensors 106 and one or more cameras 104, which are used by the sensor prediction module 124 and the camera prediction module to monitor at least one player for impacts and predict the likelihood of the player sustaining an injury from the one or more impacts. The base module 122 further uses the correlation module 128 to compare the predictions made by the sensor prediction module 124 and the camera prediction module 126 and previous data from the correlation database 120 to determine a correlated probability of injury. The base module 122 repeats until the event ends.

The sensor prediction module 124 polls at least one wearable sensor 106 for movement and impact data related to a player. The sensor prediction module 124 checks whether an impact is detected and determines an injury prediction or risk score when detected.

The sensor data is saved to a sensor database 116, and the prediction data is sent to the base module 122.

The camera prediction module 126 polls at least one camera 104 for movement and impact data related to a player within the field of view of the camera 104. The camera prediction module 126 checks whether an impact is detected and determines an injury prediction or risk score when detected. The camera data is saved to a camera database 118, and the prediction data is sent to the base module 122.

The correlation module 128 receives sensor data and camera data from the base module 122. The sensor and camera data comprise prediction data for at least one impact event detected by the sensor prediction module 124 and/or the camera prediction module 126. The correlation module 128 synchronizes the data received from the sensor prediction module 124 and the camera prediction module 126 using time codes associated with the collected data and calculating correlation coefficients. If the correlation coefficient is greater than a predetermined threshold value, it is then determined that there is a high probability of injury. If the correlation coefficient is lower than a predetermined threshold value, it is then determined that there is a low probability of injury. The data is saved to a correlation database, and the prediction data is sent to the base module 122.

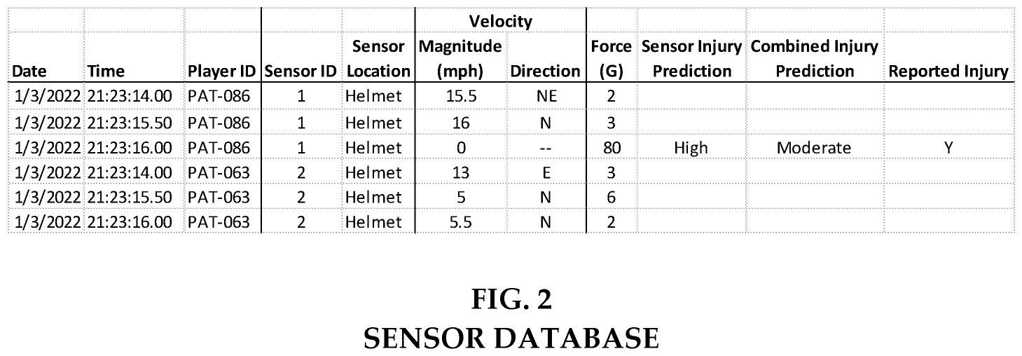

FIG. 2 illustrates the sensor database 116. The sensor database 116 comprises data collected from one or more wearable sensors 106. The data stored may include position and orientation data from sensors such as an accelerometer, GPS, compass, etc. The data may additionally comprise force data from transducers or strain gages. The data may additionally include player data such as the player wearing the sensor, the type of sensor, the sensor's location on the player, and measurements from the sensor as a specific time indicated by a timestamp. The data may also include the type of event and other players participating in the event. The sensor database 116 is populated by the sensor prediction module 124 and the correlation module 128. It is used by the sensor prediction module 124 to predict the likelihood a player has sustained an injury or risk that the player will sustain an injury in the near future, such as during continued gameplay in the current event. The sensor database 116 additionally stores the probability of injury determined by the sensor prediction module 124, the correlated or combined probability determined by the correlation database, and any reported injury. The probability may be represented by a number returned from an algorithm or may be represented by a classification, such as high or low, determined by identifying the classification into which a numerical prediction fits based upon one or more threshold values. For example, the threshold of .75 may be used such that any probability equal to or greater than .75 is classified as a high probability, and any probability lower than .75 is classified as a low probability. The data may additionally be used by the correlation module 128.

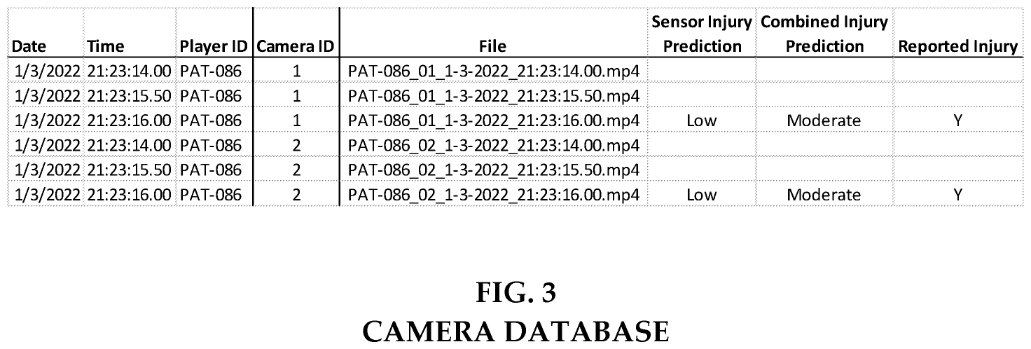

FIG. 3 illustrates the camera database 118. The camera database 118 comprises data collected from one or more cameras 104 oriented towards at least one player, or more generally, a field of play at a sporting event. A sporting event may comprise a competitive game or match or may comprise a practice session involving at least one player. The stored data may include position and orientation data from the player identified using methods such as edge detection and object recognition utilizing machine learning and artificial intelligence. The data may additionally comprise calculated force data based upon velocities and accelerations determined from camera data and estimated or predefined player and object masses. The data is populated by the camera prediction module 126 and is used by the camera prediction module 126 to predict the likelihood a player has sustained an injury or risk that the player will sustain an injury in the near future, 8such as during continued gameplay in the current event. The camera database 116 additionally stores the probability of injury determined by the camera prediction module 126, the correlated or combined probability determined by the correlation database, and any reported injury. The probability may be represented by a number returned from an algorithm or may be represented by a classification, such as high or low, determined by identifying the classification into which a numerical prediction fits based upon one or more threshold values. For example, the threshold of .75 may be used such that any probability equal to or greater than .75 is classified as a high probability, and any probability lower than .75 is classified as a low probability. The data may additionally be used by the correlation module 128.

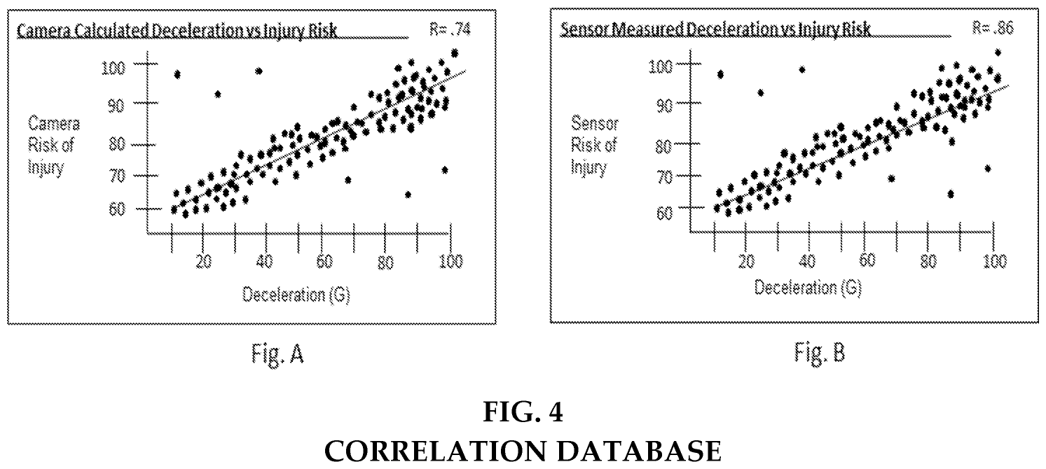

FIG. 4 illustrates the correlation database 128. The correlation database 120 stores correlations of camera data with the risk of injury as determined by the camera prediction module 126 and sensor data with the risk of injury as determined by the sensor prediction module 124. In an exemplary embodiment, the sensor and camera data is comprised of the magnitude of deceleration in Gs, or acceleration equivalent to gravity. The sensor data is measured from one or more sensors affixed to a player, such as via an accelerometer affixed to the player's helmet. The camera data is calculated from the movement of players captured over time by one or more cameras. A correlation represents the degree to which a first parameter, in this example, the measured or calculated deceleration, is related to or influences a second parameter, in this case, the predicted risk of injury. The predicted risk of injury is determined by the sensor prediction module 124 and the camera prediction module 126. In FIG. A, the correlation coefficient R is equal to .74 for deceleration calculated from camera data and the risk of injury determined by the camera prediction module 126. In FIG. B, the correlation coefficient R is equal to .86 for deceleration measured from camera data and the risk of injury determined by the sensor prediction module 124. This indicates that the sensor data and the sensor prediction module 124 is a more reliable method of predicting injury than the camera prediction module. Additionally, each correlation coefficient may be compared to a threshold value, which may be predefined or identified using machine learning or artificial intelligence to determine whether the predictions are reliable. In an example, the threshold is .75, in which case the sensor data is determined to be reliable while the camera data is determined to be unreliable. In this example, the camera data may be unreliable due to a technical issue, such as a camera requiring calibration or poor placement of the cameras.

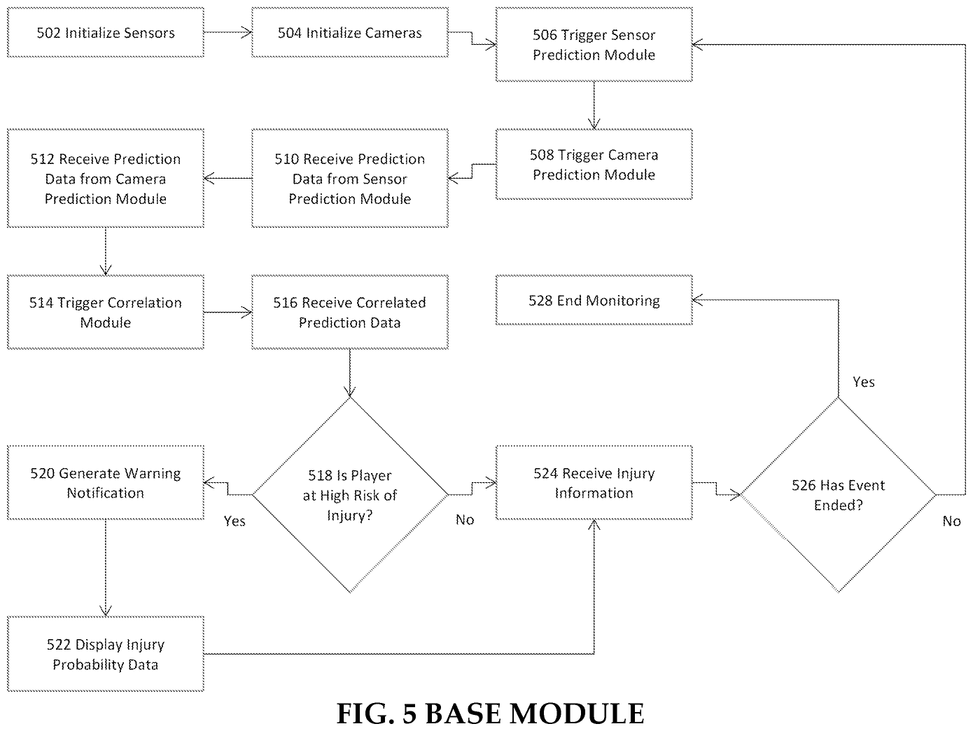

FIG. 5 illustrates the base module 122. The process begins by initializing, at step 502, the wearable sensors 106 attached to at least one player. Initialization may comprise powering on the wearable sensors 106 and may also include establishing communication between the wearable sensors 106 and a player monitoring system 102 via a communication interface 112. The communication may comprise a handshake where a message is transmitted to a wearable sensor and the wearable sensor, upon receiving the message, transmits a response that confirms that the message was received. If both messages are received, then communication is established. In an embodiment, a wearable sensor 106 is an accelerometer affixed to the helmet of an American football player, John Smith. The wearable sensor 106 is turned on, and confirmation is received by a terminal in possession of John Smith's trainer that the accelerometer is transmitting position and orientation data to the terminal. The terminal may be a mobile device such as a tablet. Initializing, at step 504, the cameras 104 oriented towards at least one player, or more generally, oriented toward the field of play. Initialization may comprise powering on the cameras 104 and may additionally include calibration to verify that each camera 104 is recording data as expected. Calibration may require a standardized card to be held in front of the camera 104 to verify color and brightness accuracy. Alternatively, the collected data from the powered-on camera 104 may be compared to previous data from the camera database 118 to determine whether the current data matches the baseline data from the camera database 118. In an embodiment, the cameras 104 are installed at an American football stadium and oriented towards the field of play. The cameras 104 are powered on, and images are acquired and compared to images retrieved from the camera database 118, previously acquired by the cameras 104, and a correlation coefficient for each camera is calculated. If the correlation coefficient is above a threshold value, 0.90, the cameras are sufficiently calibrated, and initialization is complete. The initialization process may also comprise the establishment of communication with a player monitoring system 102. Triggering, at step 506, the sensor prediction module 124. The sensor prediction module 124 polling at least one wearable sensor 106 and evaluates whether an impact is detected. The sensor prediction module 124 determines an injury prediction or risk score if an impact is detected. The acquired and calculated data is saved to the sensor database 116. Triggering, at step 508, the camera prediction module 126. The camera prediction module 126 polling at least one camera 104 and evaluates whether an impact is detected. The camera prediction module 126 determines an injury prediction or risk score if an impact is detected. The acquired and calculated data is saved to the camera database 118. Receiving, at step 510, an injury prediction from the sensor prediction module 124. The injury prediction comprises an impact force detected for player John Smith of 80 Gs detected at his helmet, which was predicted to have a high probability of having a concussion as the force is greater than a threshold value of 75 Gs. Receiving, at step 512, an injury prediction from the camera prediction module 126. The injury prediction comprising an impact force calculated for player John Smith of 60 Gs based upon analyzing the movement of John Smith in the images acquired by at least one camera 104. The camera prediction module 126 determines that John Smith has a low probability of having a concussion as the calculated force is less than a threshold value of 75 Gs. Triggering, at step 514, the correlation module 128. The correlation module 128 receives sensor data collected by the sensor prediction module 124 and camera data from the camera prediction module 126 and calculates correlation coefficients for time-synchronized data. The correlation coefficients are then compared to a threshold value to determine whether the player is at a high probability of injury or a low probability of injury based upon the correlated data. The data is further saved to the correlation database 120. Receiving, at step 516, correlated prediction data from the correlation module 128. The injury prediction comprises a high risk of injury or low risk of injury. In an embodiment, an impact detected involving John Smith is determined to have a correlation coefficient of .86, indicating a high probability of having sustained a concussion. Determining, at step 518, whether the player is at a high risk of injury. The player is at a high risk of injury if the correlated prediction data from the correlation module 128 indicates a high or moderate risk of having sustained an injury. In an embodiment, John Smith is determined to be at moderate risk of having sustained a concussion. Generating, at step 520, a warning notification is sent to any player, a coach, a trainer, or a medical provider indicating that the player has experienced an impact that has a likelihood of having resulted in an injury. The warning notification may be sent to a mobile device, such as a tablet and may comprise audio, visual, or haptic components. In an embodiment, a tablet held by the trainer for John Smith receives a notification comprising a text box on the tablet's screen, an audio notification playing an alarm tone, and a vibration to acquire the trainer's attention. Displaying, at step 522, injury probability data relating to the impact experienced by the player. The injury probability data may include any of the data collected by the wearable sensors 106, camera footage and calculated data acquired from one or more cameras 104, and probability data determined by any of the sensor prediction module 124, camera prediction module 126, or the correlation module 128. In an embodiment, the tablet held by the trainer for the player John Smith displays impact data comprised of the accelerometer data indicating an impact with a deceleration with a magnitude of 80 Gs, the camera data indicating a force of 60 Gs, and a moderate probability of injury. The data further comprises a 10-second video file comprising footage of the impact detected involving John Smith from at least one camera 104. The injury probability data may additionally comprise an error message indicating that there may be insufficient or inaccurate camera data as the sensor prediction and the camera prediction did not agree. In some embodiments, the acquired data may be used to train a machine learning model to improve camera accuracy. The error message may additionally comprise actions necessary to improve impact detection and analysis. Receiving, at step 524, injury information related to an impact event. The injury information may be provided manually from injury reports filed by the player's trainer, doctor, coach, or any other relevant personnel. The injury information may alternatively be acquired by monitoring the player's performance and comparing the player's performance before the impact event with the player's performance after the impact event. The injury being associated with either a specific impact event, such as the most recent impact event before the report of injury, or a series of impact events, such as the impact events occurring during a game before the report of injury. In an embodiment, the trainer for player John Smith reported that John Smith received a concussion related to the impact occurring at 21:23:16.00 on Jan. 3, 2022. Determining, at step 526, whether the event has ended by checking whether any playtime remains on the play clock. In the event of a practice event, the end of the event may be determined manually. Alternative embodiments include the end of the event indicated by the player of interest, all players leaving the field of play, or manually ending a monitoring application. In an embodiment, an American football game in which John Smith is playing is in the 4th quarter with 5:34 remaining on the clock; therefore, the event has not ended. In an alternate embodiment, the game clock has expired, and the score is not tied; therefore, the event has ended. Ending, at step 528, monitoring the players after the event has ended.

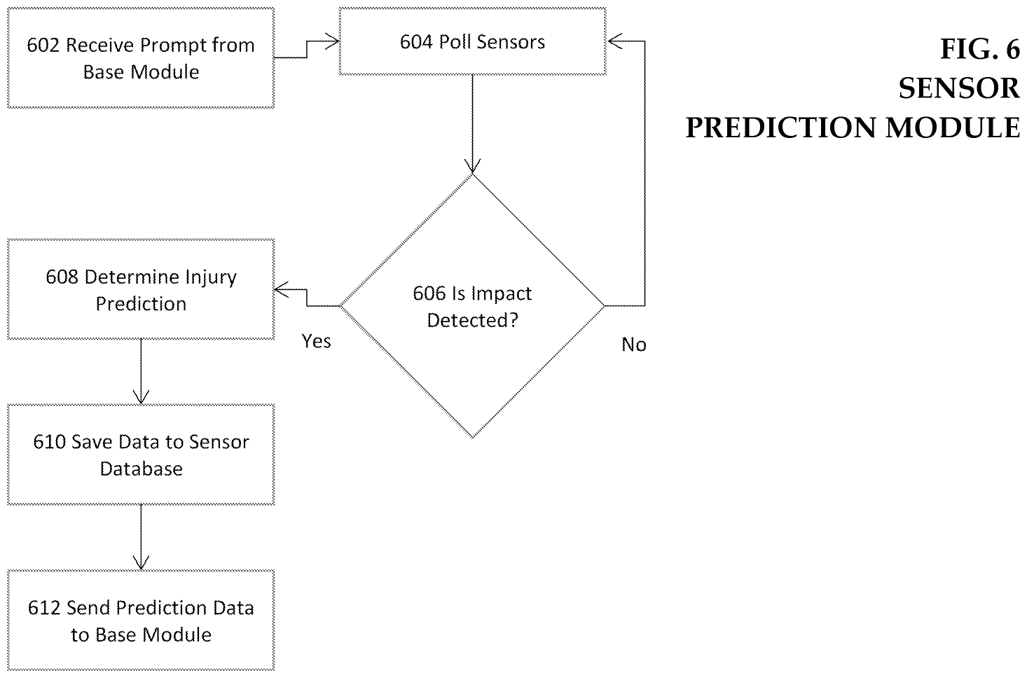

Functioning of the “Sensor Prediction Module” will now be explained with reference to FIG. 6.

FIG. 6 illustrates the sensor prediction module 124. The process begins with receiving, at step 602, a prompt from the base module 122. The sensor prediction module 124 is configured to monitor data from at least one wearable sensor 106, determine whether an impact has occurred based upon the data from at least one wearable sensor 106, and determine a probability of injury if an impact was detected. Polling, at step 604, the at least one wearable sensor 106 attached to a player. The wearable sensor 106 may be polled at a regular interval, such as every 0.5 seconds, or may transmit data as available in real-time. The sensor data may be comprised of a single measurement or multiple measurements taken over a time period, such as a measurement every five seconds for a period of one minute. Alternatively, the data may be measured in response to a trigger event, such as when movement is detected. In an embodiment, sensor data is measured by an accelerometer affixed to a helmet worn by the player, John Smith, which is transmitted to a player monitoring system 102 via a Wi-Fi connection. The accelerometer data comprises the player's location, translating to 10 feet from the right boundary at the 50-yard line, the player's velocity of 16 mph in a northerly direction, and the player's acceleration of 0.5 mph on Jan. 3, 2022, at 21:23:15.50. Determining, at step 606, whether an impact is detected from the data collected from at least one wearable sensor 106. An impact is detected if the player's velocity or acceleration falls abruptly or their direction changes at a rate exceeding a threshold, which may indicate a side impact from another player. In an embodiment, the player, John Smith, is moving at 16 mph in a northerly direction on Jan. 3, 2022, at 21:23:15.50. A half-second later, at 21:23:16.00, John Smith is moving at 0 mph with a deceleration detected with a magnitude of 80 Gs or 80 times the acceleration of gravity transmitted from an accelerometer located in his helmet. Determining, at step 608, a prediction that the player has sustained an injury from the detected impact. The prediction may be based upon a predetermined threshold value, such as a head impact exceeding 75 Gs may indicate a high likelihood of a concussion. These threshold values may be static and comprise a set of reference threshold values depending on the location and magnitude of the impact. In alternate embodiments, a machine learning algorithm or artificial intelligence may be utilized to predict the likelihood of injury. An artificial intelligence may use a model created using past wearable sensor 106 data from the sensor database 116 to predict the likelihood of injury. Further, the type of injury may also be predicted. In an embodiment, player John Smith is determined to have a high risk of concussion from the impact as the deceleration of magnitude 80 Gs is greater than the threshold value of 75 Gs. In alternate embodiments, the likelihood of injury may refer to the player's susceptibility to future injury. For example, the wearable sensor 106 data for an impact with a magnitude of 60 Gs may indicate a low likelihood of having sustained a concussion, as the force is less than the threshold value of 75 Gs; however, the player may be determined to have a high likelihood of injury within the next hour if they continue to play based upon the data collected by the wearable sensors 106. Saving, at step 610, the data collected by the wearable sensors 106 and the prediction of the likelihood of injury to the sensor database 116. In an example, on Jan. 3, 2022, at 21:23:15.50, John Smith is moving at 16 mph in a northerly direction, and at 21:23:16.00, John smith is moving at 0 mph and is determined to have been subjected to a force of 80 Gs at his head. Further saving the determined high risk of a concussion, sending, at step 612, the injury prediction data to the base module 122. The injury prediction data comprises that player John Smith was subjected to a force of 80 Gs and has a high risk of a concussion.

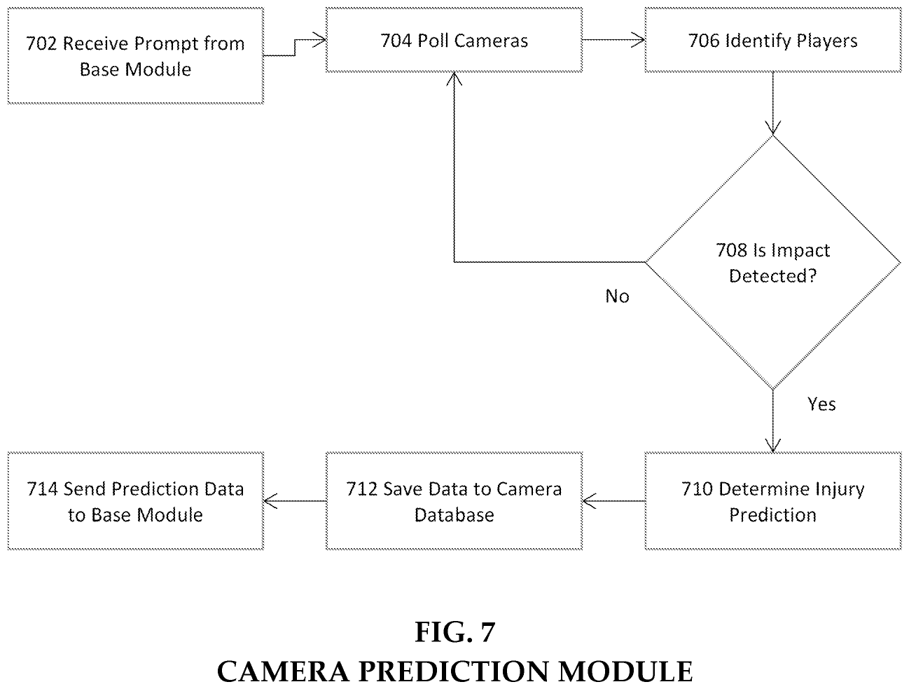

Functioning of the “Camera Prediction Module” will now be explained with reference to FIG. 7.

FIG. 7 illustrates the camera prediction module 126. The process begins with receiving, at step 702, a prompt from the base module 122. The camera prediction module 126 being configured to monitor data from at least one camera 104, determine whether an impact has occurred involving the player, and determine a probability of injury if an impact was detected. Polling, at step 704, the at least one camera 104 oriented towards a player or a field of play. The camera 104 may be polled at a regular interval, such as every 0.1 seconds, or the camera's maximum framerate in real-time. The camera data may comprise a single image frame or a video taken over a time period, such as in 10-second intervals. In an embodiment, a camera oriented toward an American football field captures the movement of John Smith and a multitude of other players during a football game. Identifying, at step 706, players on the field of play within the field of view of at least one camera 104. The players may be identified using any of facial recognition, object detection, edge detection, etc. Alternatively, the cameras 104 may operate in conjunction with other sensors or systems to track the position of players on the field of play to track their position. In an embodiment, edge detection is used to create a shape representing the outline of each player, and object detection is used to identify the player based on their uniform indicating the team they play on and their player number. This identifying information may be stored in the camera database 118 to identify players. In an embodiment, identifying a player John Smith as a player for the New England Patriots with the number 86. In addition to identifying players, the camera prediction module 126 may identify objects that one or more players may collide with, using edge detection and object recognition. Detecting, at step 708, whether an impact has been detected. An impact is detected when two or more players, or at least one player and an object on the field of play, make contact within the field of view of at least one camera 104. In an embodiment, the shape identified for player John Smith contacts with the shape for another player, and therefore an impact is determined to have occurred. In some embodiments, a second image from a second camera 104 may be required to determine whether an impact has occurred in three-dimensional space. Determining that an impact has been detected may further comprise the calculation of velocities for each player involved and then determining an acceleration or force. A player's velocity can be determined by taking the player's distance traveled between image frames and dividing by the time elapsed between the capture of the image frames. An impact may then be defined as contact between two or more players where at least one player was moving at a speed greater than a threshold value, such as five mph, or acceleration experienced by at least one player exceeds 1 G. In an embodiment, player John Smith is detected to have experienced an impact with acceleration with a magnitude of 60 Gs. Determining, at step 710, a prediction that the player has sustained an injury from the detected impact. The prediction may be based upon a predetermined threshold value, such as a head impact exceeding 75 Gs may indicate a high likelihood of a concussion. These threshold values may be static and comprise a set of reference threshold values depending on the location and magnitude of the impact. In alternate embodiments, a machine learning algorithm or artificial intelligence may be utilized to predict the likelihood of injury. An artificial intelligence may use a model created using past camera 104 data from the camera database 118 to predict the likelihood of injury. Further, the type of injury may also be predicted. In an embodiment, player John Smith is determined to have a low risk of concussion from the impact as the calculated deceleration of a magnitude 60 Gs is less than the threshold value of 75 Gs. In an alternate embodiment, the likelihood of injury may refer to the player's susceptibility to future injury. For example, John Smith's impact with a magnitude of 60 Gs at the head may indicate a low likelihood of having sustained a concussion, as the force is less than the threshold value of 85 Gs; however, he may be determined to have a high likelihood of injury within the next hour if he continues to play based upon the data collected by the camera 104. Saving, at step 712, the data collected by the cameras 104 and the prediction of the likelihood of injury to the camera database 118. In an example, on Jan. 3, 2022, at 21:23:15.50, John Smith is moving at 13 mph in a northerly direction, and at 21:23:16.00, John Smith is moving at 0 mph and is determined to have been subjected to a force of 60 Gs at his head. Further saving the determined low risk of a concussion. Sending, at step 714, the injury prediction data to the base module 122. The injury prediction data comprises that player John Smith was subjected to a force of 60 Gs and has a low risk of a concussion.

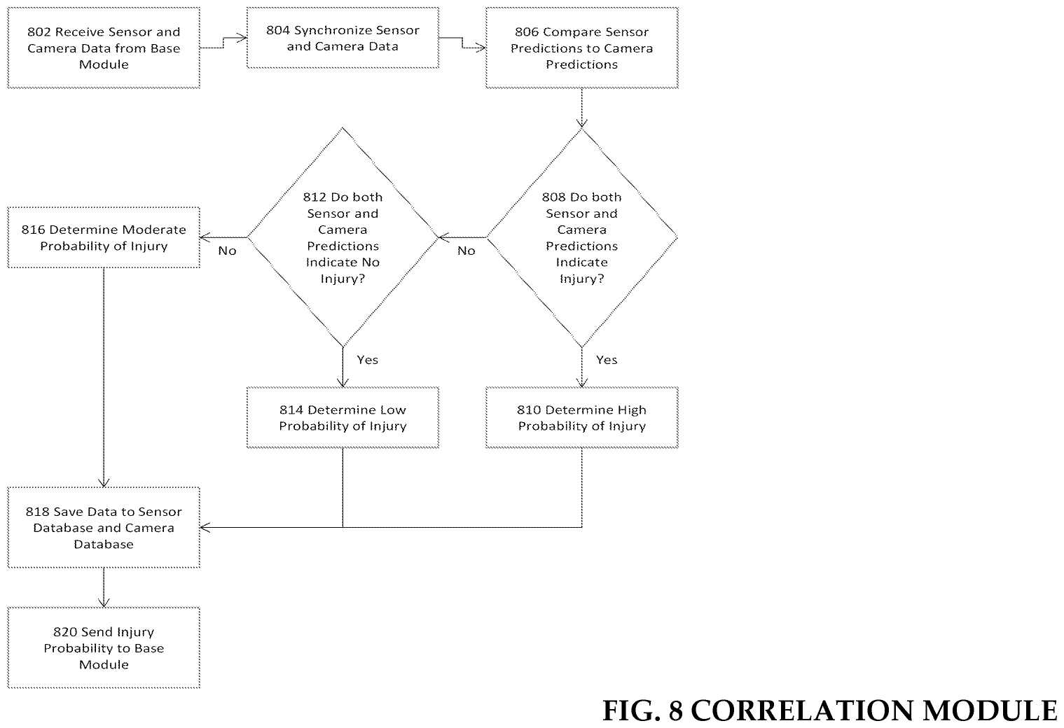

Functioning of the “Correlation Module” will now be explained with reference to FIG. 8.

FIG. 8 illustrates the correlation module 128. The process begins with receiving, at step 802, sensor and camera data from the base module 122. The sensor and camera data comprise data collected by the sensor prediction module 124 and the camera prediction module 126, including prediction data. In an embodiment, the sensor data comprising John Smith moving at 16 mph in a northerly direction at 21:23:15.50 and then moving at a speed of 0 mph at 21:23:16.00 after being subjected to a deceleration of a magnitude of 80 Gs measured by an accelerometer mounted on his helmet. The sensor data further comprises a high risk of concussion. The camera data comprises John Smith moving at a calculated 13 mph in a northerly direction at 21:23:15.50 and then moving at a speed of 0 mph at 21:23:16.00, having experienced a calculated deceleration of 60 Gs. The camera data further comprised a low risk of concussion. Synchronizing, at step 804, sensor and camera data such that the time stamps for the sensor data and the camera data match or are the closes available. In an embodiment, the time stamp for sensor data is 21:23:15.50, and the time stamp for the camera data is also 21:23:15.50; therefore, the data is synchronized. In an alternate embodiment, the sensor data detected an impact at 21:23:15.50; however, the camera data detected an impact at 21:23:17.00. The data is synchronized by retrieving the camera data for the time of the impact detected by at least one wearable sensor 106 at the time 21:23:15.50. Alternatively, retrieving the sensor data for the time of the impact detected by at least one camera 104 at the time 21:23:17.00. In an embodiment, the impact may be evaluated at the impact times detected by both the sensor prediction module 124 and the camera prediction module 126 if they are different, or the impact may be evaluated over a period of time defined by the earliest detection of impact through the latest detection of impact. Comparing, at step 806, the sensor predictions and the camera predictions for a synchronized impact event. The predictions may be comprised of a probability that the player received an injury from the detected impact. Alternatively, the predictions may comprise a risk score which may be determined by multiplying the prediction as a percentage by a confidence interval to get a weighted score. In an embodiment, the confidence interval is the correlation coefficient stored in the correlation database. In alternate embodiments, the predictions may be classified based on a threshold value into classifications such as high or low. In an embodiment, the sensor prediction of injury is high, and the camera prediction is low. Determining, at step 808, whether both the sensor predictions and the camera predictions indicate a high probability of injury. A high probability of injury may comprise risk scores above a threshold value. Alternatively, a high probability may be determined by the sensor prediction module 124 and the camera prediction module 126. In an embodiment, the sensor prediction of injury is high, and the camera prediction is low. Determining, at step 810, that there is a high correlated probability of injury if both the sensor predictions and the camera predictions indicate a high likelihood of injury. In an embodiment, the sensor prediction of injury is high, and the camera prediction of injury is low; therefore, there is not a high probability of injury. In an alternate embodiment, the sensor prediction of injury is high, and the camera prediction of injury is also high; therefore, there is a high probability of injury. Determining, at step 812, whether both the sensor predictions and the camera predictions indicate a low probability of injury. A low probability of injury may comprise risk scores below a threshold value. Alternatively, a low probability may be determined by the sensor prediction module 124 and the camera prediction module 126. In an embodiment, the sensor prediction of injury is high, and the camera prediction is low. Determining, at step 814, that there is a low correlated probability of injury if both the sensor predictions and the camera predictions indicate a low likelihood of injury. In an embodiment, the sensor prediction of injury is high, and the camera prediction of injury is low; therefore, there is not a low probability of injury. In an alternate embodiment, the sensor prediction of injury is low, and the camera prediction of injury is also low; therefore, there is a low probability of injury. Determining, at step 816, that there is a moderately correlated probability of injury if only one of the sensor predictions or the camera predictions indicates a high probability of injury and the other indicates a low probability of injury. In this case, where the predictions made by the sensor prediction module 124 and the camera prediction module 126 do not agree, the probability of injury is determined to be moderate, as the data does not support a high or low correlated probability of injury. This is likely indicative of a inacurate camera data, poor camera angle, etc. In an embodiment, the camera and sensor data may be used to train a machine learning model to improve impact detection and analysis of the camera data. The updated model may be saved to the correlation database 120. Saving, at step 818, the correlated probability of injury to the sensor database 116 and the camera database 118. The correlated probability of injury may comprise a composite risk score, such as an average risk score comprising the sensor risk score and the camera risk score. Alternatively, the correlated probability of injury may be a classification, such as high, moderate, or low. The correlated probability of injury provides for higher confidence of the determined low or high probability of injury or identifies possible failures within the system to identify possible false positive or false negative determinations. Sending, at step 816, the correlated injury prediction to the base module 122. The correlated injury prediction comprises a moderate probability that the player, John Smith, has a high likelihood of having a concussion as both sensor and camera data indicate a probability of injury above a threshold value. The moderate probability of injury may also comprise an error message indicating the system may require maintenance as the sensor and camera predictions were not in agreement.

The functions performed in the processes and methods may be implemented in differing order. Furthermore, the outlined steps and operations are only provided as examples. Some of the steps and operations may be optional, combined into fewer steps and operations, or expanded into additional steps and operations without detracting from the essence of the disclosed embodiments.

1. A method of predicting injury resulting from an impact during a physical activity comprising:
a sensor prediction module executable to monitor at least one wearable sensor and determining a probability of injury resulting from an impact based on the wearable sensor data;
a camera prediction module executable to monitor at least one camera and determining a probability of injury resulting from an impact based on the camera data; and
a correlation module executable to correlate the probability of injury resulting from an impact based on the sensor data with the probability of injury resulting from an impact based on the camera data, wherein a probability of injury is determined based on the correlation of the probability of injury determined by the sensor prediction module and the camera prediction module.
